(19) United States
(12) Patent Application Publication
(10) Pub. No.: US 2023/0323473 A1
(43) Pub. Date: Oct. 12, 2023

(54) METHODS FOR MULTIMODAL EPIGENETIC SEQUENCING ASSAYS

(71) Applicants: Helio Health Inc., Irvine, CA (US); The Regents of the University of California, Oakland, CA (US)

(72) Inventors: Wei LI, Irvine, CA (US); Yumei LI, Irvine, CA (US); Jianfeng XU, Beijing (CN); David TAGGART, West Lafayette, IN (US)

(73) Assignees: Helio Health Inc., Irvine, CA (US); The Regents of the University of California, Oakland, CA (US)

(21) Appl. No.: 18/116,196

(22) Filed: Mar. 1, 2023

Related U.S. Application Data
(60) Provisional application No. 63/316,277, filed on Mar. 3, 2022.

(51) Int. Cl. C12Q 1/6886

(52) U.S. Cl. CPC C12Q 1/6886; C12Q 1/6869; C12Q 2600/154

(57) ABSTRACT

Provided herein, in certain aspects, are methods involving epigenetic signatures comprising features from any of a methylation profile, a nucleosome dynamics profile, or a fragmentation profile, or any combination thereof. In other aspects, the present disclosure is directed to methods involving an epigenetic signature (such as methods of determining an epigenetic signature, methods of discovering an epigenetic signature, methods of diagnosis, and methods and treatment), and system, kits, and components useful therefor.

### CROSS REFERENCE TO RELATED APPLICATIONS

This application claims priority benefit of U.S. Provisional Application No. 63/316,277, filed on Mar. 3, 2022, the contents of which are incorporated herein by reference in its entirety.

### TECHNICAL FIELD

The present disclosure, in certain aspects, is directed to multimodal epigenetic signatures comprising features from any of a methylation profile, a nucleosome dynamics profile, or a fragmentation profile, or any combination thereof. In other aspects, the present disclosure is directed to methods involving said epigenetic signature, and system, kits, and components useful therefor.

### BACKGROUND

Techniques for non-invasively detecting a biological state of an individual, such as a disease state and/or response to treatment, are highly desirable. Under normal conditions, nucleic acids are shed into systemic circulation via, e.g., apoptosis, and circulate as cell-free nucleic acids such as cell-free DNA (cfDNA). Nucleic acids may also be shed into systemic circulation due to or originating from diseased cells, such as cancerous cells. CfDNA has been a source of non-invasively obtained biological material for studying a biological state of an individual. However, it remains a great challenge to identify relevant and robust cfDNA markers to detect a biological state of an individual.

### BRIEF SUMMARY

In some aspects, provided herein is a method of determining an epigenetic signature from a sample obtained from an individual, the method comprising analyzing data obtained from a non-disruptive methylation sequencing technique performed on the sample obtained from the individual to determine the epigenetic signature, wherein the epigenetic signature comprises features obtained from two or more of the following profiles: a methylation profile comprising information derived from one or more methylation sites; a nucleosome dynamics profile comprising information derived from any one or more of: (a) nucleosome positional information; (b) nucleosome occupancy; or (c) nucleosome fuzziness; or a fragmentation profile comprising information derived from read distributions in one or more base length windows.

In some embodiments, provided herein is a method of determining an epigenetic signature from a sample obtained from an individual, the method comprising analyzing data obtained from a methylation sequencing technique performed on the sample obtained from the individual to determine the epigenetic signature, wherein the epigenetic signature comprises features obtained from two or more of the following profiles: a methylation profile comprising information derived from one or more methylation sites; a nucleosome dynamics profile comprising information derived from any one or more of: (a) nucleosome positional information; (b) nucleosome occupancy; or (c) nucleosome fuzziness; or a fragmentation profile comprising information derived from read distributions in one or more base length windows.

In some aspects, provided herein is a method of generating an epigenetic signature from a sample obtained from an individual, the method comprising: receiving sequencing data obtained from a non-disruptive methylation sequencing technique performed on the sample obtained from the individual; extracting features from the sequencing data, wherein the features include information from two or more of the following profiles: a methylation profile comprising information derived from one or more methylation sites; a nucleosome dynamics profile comprising information derived from any one or more of: (a) nucleosome positional information; (b) nucleosome occupancy; or (c) nucleosome fuzziness; or a fragmentation profile comprising information derived from read distributions in one or more base length windows; inputting the extracted features into a machine learning model; analyzing the features using the machine learning model to generate the epigenetic signature based on a plurality of the features; and outputting the generated epigenetic signature.

In some embodiments, provided herein is a method of generating an epigenetic signature from a sample obtained from an individual, the method comprising: receiving sequencing data obtained from a methylation sequencing technique performed on the sample obtained from the individual; extracting features from the sequencing data, wherein the features include information from two or more of the following profiles: a methylation profile comprising information derived from one or more methylation sites; a nucleosome dynamics profile comprising information derived from any one or more of: (a) nucleosome positional information; (b) nucleosome occupancy; or (c) nucleosome fuzziness; or a fragmentation profile comprising information derived from read distributions in one or more base length windows; inputting the extracted features into a machine learning model; analyzing the features using the machine learning model to generate the epigenetic signature based on a plurality of the features; and outputting the generated epigenetic signature.

In some aspects, provided herein is a method of diagnosing a disease in an individual, the method comprising: determining an epigenetic signature from data obtained from a non-disruptive methylation sequencing technique performed on a sample obtained from the individual, wherein the epigenetic signature comprises features obtained from two or more of the following profiles: a methylation profile comprising information derived from one or more methylation sites; a nucleosome dynamics profile comprising information derived from any one or more of: (a) nucleosome positional information; (b) nucleosome occupancy; or (c) nucleosome fuzziness; or a fragmentation profile comprising information derived from read distributions in one or more base length windows; and diagnosing the disease in the individual based on the epigenetic signature as compared to a disease epigenetic signature.

In some embodiments, provided herein is a method of diagnosing a disease in an individual, the method comprising: determining an epigenetic signature from data obtained from a methylation sequencing technique performed on a sample obtained from the individual, wherein the epigenetic signature comprises features obtained from two or more of the following profiles: a methylation profile comprising information derived from one or more methylation sites; a nucleosome dynamics profile comprising information derived from any one or more of: (a) nucleosome positional information; (b) nucleosome occupancy; or (c) nucleosome fuzziness; or a fragmentation profile comprising information derived from read distributions in one or more base length windows; and diagnosing the disease in the individual based on the epigenetic signature as compared to a disease epigenetic signature.

In some embodiments, the method further comprises diagnosing a disease in the individual based on the epigenetic signature as compared to a disease epigenetic signature. In some embodiments, provided herein is a method of treating a disease in an individual, the method comprising: diagnosing the individual as having the disease according to methods of diagnosing a disease in an individual provided herein; and administering an agent to treat the disease in the individual.

In some aspects, provided herein is a method of identifying a disease epigenetic signature indicative of an individual having a disease, the method comprising: receiving sequencing data from a plurality of individuals having the disease and a plurality of individual not having the disease, wherein the sequencing data is obtained from a non-disruptive methylation sequencing technique performed on samples obtained from the individuals; extracting features from the sequencing data, wherein the features include information from two or more of the following profiles: a methylation profile comprising information derived from one or more methylation sites; a nucleosome dynamics profile comprising information derived from any one or more of: (a) nucleosome positional information; (b) nucleosome occupancy; or (c) nucleosome fuzziness; or a fragmentation profile comprising information derived from read distributions in one or more base length windows; inputting the extracted features into a machine learning model, wherein the extracted features from each of the plurality of individuals are embedded with an associated classification of the individual having the disease or not having the disease; training the machine learning model using the extracted features to identify the disease epigenetic signature; and outputting the disease epigenetic signature.

In some aspects, provided herein is a method of identifying a disease epigenetic signature indicative of an individual having a disease, the method comprising: receiving sequencing data from a plurality of individuals having the disease and a plurality of individual not having the disease, wherein the sequencing data is obtained from a methylation sequencing technique performed on samples obtained from the individuals; extracting features from the sequencing data, wherein the features include information from two or more of the following profiles: a methylation profile comprising information derived from one or more methylation sites; a nucleosome dynamics profile comprising information derived from any one or more of: (a) nucleosome positional information; (b) nucleosome occupancy; or (c) nucleosome fuzziness; or a fragmentation profile comprising information derived from read distributions in one or more base length windows; inputting the extracted features into a machine learning model, wherein the extracted features from each of the plurality of individuals are embedded with an associated classification of the individual having the disease or not having the disease; training the machine learning model using the extracted features to identify the disease epigenetic signature; and outputting the disease epigenetic signature.

In some embodiments, each of the one or more methylation sites of the methylation profile are selected from the group consisting of cg18081940, cg23089825, cg16395183, cg19811148, cg07790615, cg20996351, cg04977528, cg24465685, cg20428713, cg13678973, cg25339566, cg16596317, cg23786625, cg11328303, cg19578660, cg02272851, cg10298052, cg13585930, cg23575688, cg12394201, cg08149193, cg18854419, cg07603330, cg10658542, cg13099890, cg22302985, cg13596497, cg14507533, cg25366582, cg22396555, cg10566012, cg05168229, cg10795666, cg25078444, cg16038120, cg23883632, cg18380808, cg13615592, cg00250422, cg19691260, cg16558770, cg15681853, cg03397724, cg10514097, cg06674117, cg16047279, cg12127472, cg08843809, cg08697732, cg06384763, cg04203646, cg17112426, cg08278741, cg14587524, cg26087117, cg18320766, cg08063125, cg10004780, cg18921980, cg02514318, cg20002504, cg18897632, cg15313459, cg19370054, cg16564824, cg02631468, cg01471196, cg23770904, cg18412834, cg24080247, cg11549874, cg13155421, cg19442495, cg22536150, cg05413061, cg23346462, cg09477895, cg13605674, cg13314965, cg09417547, cg00181669, cg23967169, cg10237419, cg21077559, cg27600205, cg19755714, cg18797590, cg00699993, cg06485940, cg27661394, cg00939495, cg11036833, cg23915769, cg07224726, cg02022733, cg03640756, cg15361590, cg04598517, cg06782035, cg13954457, cg25482900, cg20952257, cg14062050, cg01881524, cg11538641, cg11387340, cg05389236, cg19419054, cg10575547, cg17240815, cg24772267, cg00920327, cg00772257, cg26253500, cg23244488, cg22778435, cg26065247, cg02088996, cg19868631, cg22280038, cg07803375, cg20230721, cg03333330, cg21517947, cg10406295, cg05166490, cg07739205, cg20980783, cg06617456, cg01568998, cg13407456, cg23758305, cg20675505, cg07585876, cg03734437, and cg13410764.

In some embodiments, the one or more methylation sites of the methylation profile comprise one or more gene promoter region methylation sites.

In some embodiments, the methylation profile comprises quantitative information from at least one of the one or more methylation sites. In some embodiments, the quantitative information is based on a β-value from the at least one methylation sites. In some embodiments, the quantitative information is based on a CHALM ratio from the at least one methylation sites.

In some embodiments, the nucleosome dynamics information is based on a nucleosome at a genomic locus. In some embodiments, the nucleosome positional information is based on a window protection score (WPS). In some embodiments, the WPS is an average WPS. In some embodiments, the nucleosome occupancy is based on the frequency a nucleosome occupies a genomic region. In some embodiments, the nucleosome occupancy is obtained via normalized read coverage measured by counts per million. In some embodiments, the nucleosome fuzziness is based on the deviation of a nucleosome position from a prefer nucleosome position. In some embodiments, the fragmentation profile is based on one or more base length windows occupying the range of 30 to 250 bases in length. In some embodiments, the base length window is at least 10 bases in length. In some embodiments, the nucleosome dynamic information is obtained via DANPOS.

In some embodiments, the epigenetic signature is indicative of whether the individual has a disease. In some embodiments, the epigenetic signature comprises features from the methylation profile and the nucleosome dynamics profile. In some embodiments, the epigenetic signature comprises features from the methylation profile and the fragmentation profile. In some embodiments, the epigenetic signature comprises features from the nucleosome dynamics profile and the fragmentation profile. In some embodiments, the epigenetic signature comprises features from the methylation profile, the nucleosome dynamics profile, and the fragmentation profile. In some embodiments, the nucleosome dynamics profile comprises information derived from nucleosome positional information. In some embodiments, the nucleosome dynamics profile comprises information derived from nucleosome occupancy. In some embodiments, the nucleosome dynamics profile comprises information derived from nucleosome fuzziness.

In some embodiments, the non-disruptive methylation sequencing technique is an EM-seq technique. In some embodiments, the non-disruptive methylation sequencing technique is performed based on targeted genetic locations. In some embodiments, the method further comprises performing the non-disruptive methylation sequencing technique.

In some embodiments, the data obtained from the non-disruptive methylation sequencing technique comprises a plurality of sequence reads. In some embodiments, the method further comprises processing the plurality of sequence reads to remove low-quality reads and/or remove adaptor contamination and/or filter based on sequence read size. In some embodiments, the method further comprises aligning the plurality of sequence reads with a reference genome.

In some embodiments, the machine learning model comprises a support vector machine model, a random forest machine model, or a logistic regression machine model. In some embodiments, the method further comprises a cross-validation procedure.

In some embodiments, the sample is a cell-free DNA sample. In some embodiments, the method further comprises obtaining the sample.

In some embodiments, the disease is a cancer. In some embodiments, the cancer is a colorectal cancer. In some embodiments, the individual is a human. In some embodiments, the individual is suspected of having a disease.

### BRIEF DESCRIPTION OF THE DRAWINGS

FIG. 1 shows an exemplary workflow 100 schematic for certain methods provided herein.

### DETAILED DESCRIPTION

Provided herein, in certain aspects, are multimodal epigenetic signatures comprising features obtained from any combination of two or more of a methylation profile, a nucleosome dynamics profile (including any features thereof such as nucleosome positional information, nucleosome occupancy, and nucleosome fuzziness), and a fragmentation profile, and multimodal methods of use thereof. The disclosure of the present application is based on the inventors' unique perspective and unexpected findings regarding multimodal analyses that provide significant improvements in the determination of a state of an individual, such as a disease state, using the epigenetic signatures and methods taught herein. Specifically, the inventors have developed flexible methods for using non-disruptive methylation sequencing techniques to obtain information to generate any combination of a methylation profile, a nucleosome dynamics profile, and a fragmentation profile from a single assay. Paired with machine learning techniques, the description herein provides unexpectedly flexible, accurate, sensitive, and robust measures of a biological state of an individual. For example, the inventors demonstrated that an epigenetic signature comprising a methylation profile and a nucleosome dynamics profile provided significantly improved sensitivity for the detection of colon cancer (see Example 1). Due to the flexibility provided by the multimodal epigenetic signatures provided herein, such findings can be expanded to a diverse array of human diseases having different epigenetic footprints.

Thus, in some aspects, provided herein is a method for determining an epigenetic signature from a sample obtained from an individual, the method comprising analyzing data obtained from a non-disruptive methylation sequencing technique performed on the sample obtained from the individual to determine the epigenetic signature, wherein the epigenetic signature comprises features obtained from two or more of the following profiles: a methylation profile comprising information derived from one or more methylation sites; a nucleosome dynamics profile comprising information derived from any one or more of: (a) nucleosome positional information; (b) nucleosome occupancy; or (c) nucleosome fuzziness; or a fragmentation profile comprising information derived from read distributions in one or more base length windows.

In some aspects, provided herein is a method for determining an epigenetic signature from a sample obtained from an individual, the method comprising analyzing data obtained from a methylation sequencing technique performed on the sample obtained from the individual to determine the epigenetic signature, wherein the epigenetic signature comprises features obtained from two or more of the following profiles: a methylation profile comprising information derived from one or more methylation sites; a nucleosome dynamics profile comprising information derived from any one or more of: (a) nucleosome positional information; (b) nucleosome occupancy; or (c) nucleosome fuzziness; or a fragmentation profile comprising information derived from read distributions in one or more base length windows.

In some aspects, provided herein is a method for determining an epigenetic signature from a sample obtained from an individual, the method comprising analyzing data obtained from a non-disruptive methylation sequencing technique and one or more additional sequencing techniques (e.g., deep sequencing) performed on the sample obtained from the individual to determine the epigenetic signature, wherein the epigenetic signature comprises features obtained from two or more of the following profiles: a methylation profile comprising information derived from one or more methylation sites; a nucleosome dynamics profile comprising information derived from any one or more of: (a) nucleosome positional information; (b) nucleosome occupancy; or (c) nucleosome fuzziness; or a fragmentation profile comprising information derived from read distributions in one or more base length windows.

In other aspects, provided herein is a method for generating an epigenetic signature from a sample obtained from an individual, the method comprising: receiving sequencing data obtained from a non-disruptive methylation sequencing technique performed on the sample obtained from the individual; extracting features from the sequencing data, wherein the features include information from two or more of the following profiles: a methylation profile comprising information derived from one or more methylation sites; a nucleosome dynamics profile comprising information derived from any one or more of: (a) nucleosome positional information; (b) nucleosome occupancy; or (c) nucleosome fuzziness; or a fragmentation profile comprising information derived from read distributions in one or more base length windows; inputting the extracted features into a machine learning model; analyzing the features using the machine learning model to generate the epigenetic signature based on a plurality of the features; and outputting the generated epigenetic signature.

In other aspects, provided herein is a method for diagnosing a disease in an individual, the method comprising: determining an epigenetic signature from data obtained from a non-disruptive methylation sequencing technique performed on a sample obtained from the individual, wherein the epigenetic signature comprises features obtained from two or more of the following profiles: a methylation profile comprising information derived from one or more methylation sites; a nucleosome dynamics profile comprising information derived from any one or more of: (a) nucleosome positional information; (b) nucleosome occupancy; or (c) nucleosome fuzziness; or a fragmentation profile comprising information derived from read distributions in one or more base length windows; and diagnosing the disease in the individual based on the epigenetic signature as compared to a disease epigenetic signature. In some embodiments, the method further comprises diagnosing a disease in the individual based on the epigenetic signature as compared to a disease epigenetic signature.

In other aspects, provided herein is a method of treating a disease in an individual, the method comprising: diagnosing the individual as having the disease according to any claim herein; and administering an agent to treat the disease in the individual.

In other aspects, provided herein is a method for identifying a disease epigenetic signature indicative of an individual having a disease, the method comprising: receiving sequencing data from a plurality of individuals having the disease and a plurality of individual not having the disease, wherein the sequencing data is obtained from a non-disruptive methylation sequencing technique performed on samples obtained from the individuals; extracting features from the sequencing data, wherein the features include information from two or more of the following profiles: a methylation profile comprising information derived from one or more methylation sites; a nucleosome dynamics profile comprising information derived from any one or more of: (a) nucleosome positional information; (b) nucleosome occupancy; or (c) nucleosome fuzziness; or a fragmentation profile comprising information derived from read distributions in one or more base length windows; inputting the extracted features into a machine learning model, wherein the extracted features from each of the plurality of individuals are embedded with an associated classification of the individual having the disease or not having the disease; training the machine learning model using the extracted features to identify the disease epigenetic signature; and outputting the disease epigenetic signature.

### A. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For instance, where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure. In some embodiments, two opposing and open ended ranges are provided for a feature, and in such description it is envisioned that combinations of those two ranges are provided herein. For example, in some embodiments, it is described that a feature is greater than about 10 units, and it is described (such as in another sentence) that the feature is less than about 20 units, and thus, the range of about 10 units to about 20 units is described herein.

The term “about” as used herein refers to the usual error range for the respective value readily known in this technical field. Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise. For example, “a” or “an” means “at least one” or “one or more.” It is understood that aspects and variations described herein include embodiments “consisting” and/or “consisting essentially of” such aspects and variations.

As used herein, a “subject” or an “individual,” which are terms that are used interchangeably, is a mammal. In some embodiments, a “mammal” includes humans, non-human primates, domestic and farm animals, and zoo, sports, or pet animals, such as dogs, horses, rabbits, cattle, pigs, hamsters, gerbils, mice, ferrets, rats, cats, monkeys, etc. In some embodiments, the subject or individual is human.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of a pathological consequence of the disease.

The methods of the invention contemplate any one or more of these aspects of treatment. Those skilled in the art will recognize that several embodiments are possible within the scope and spirit of the present disclosure. The following description illustrates the disclosure and, of course, should not be construed in any way as limiting the scope of the inventions described herein.

### B. Methods Associated With the Multimodal Epigenetic Signatures Provided Herein

In certain aspects, provided herein are methods associated with the multimodal epigenetic signature taught herein comprising features obtained from any combination of two or more of the following profiles: a methylation profile comprising information derived from one or more methylation sites; a nucleosome dynamics profile comprising information derived from any one or more of: (a) nucleosome positional information; (b) nucleosome occupancy; or (c) nucleosome fuzziness; or a fragmentation profile comprising information derived from read distributions in one or more base length windows. In some embodiments, the term multimodal as used herein refers to the combination of two or more different profiles, including a methylation profile, a nucleosome dynamics profile, and a fragmentation profile, in the described methods and epigenetic signatures. The two or more different profiles may be combined to result in an improved technique, such as by a machine learning technique and cross validation.

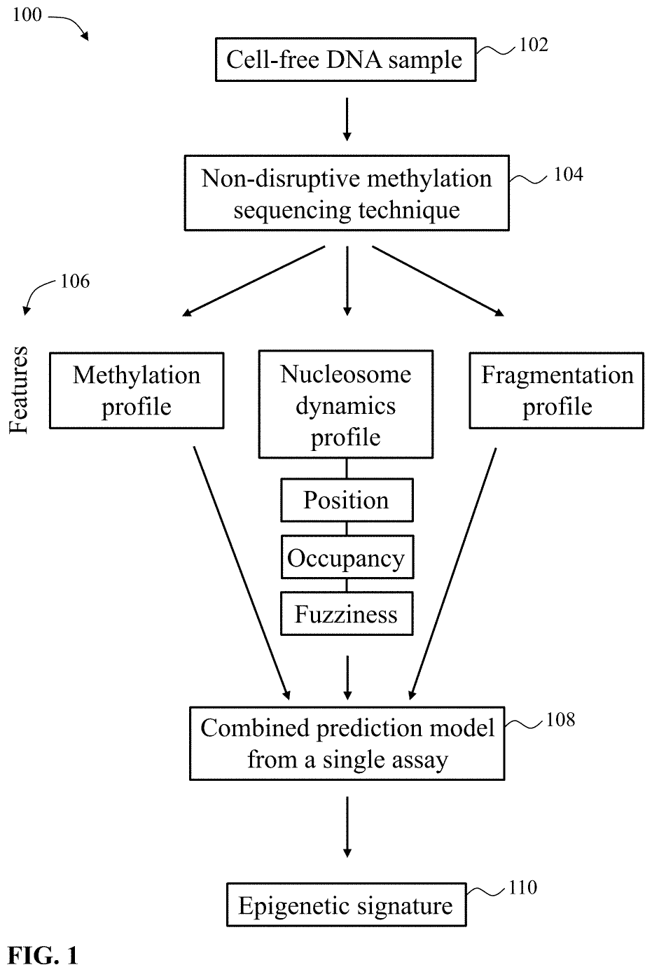

For purposes of illustrating the description provided herein, an exemplary workflow 100 schematic is provided in FIG. 1. As shown, in some embodiments, the workflow 100 begins with a cell-free DNA (cfDNA) sample 102. Such sample may be obtained from a blood sample obtained from an individual, such as an individual being assessed for a disease, and further sample processing may occur to obtain or study the cfDNA sample. The cfDNA sample is then subjected to a non-disruptive methylation sequencing technique 104, such as EM-seq. Sequencing information obtained from the non-disruptive methylation sequencing technique 104 can then be analyzed based on any configuration of desired multimodal features 106, including any of a methylation profile, a nucleosome dynamics profile, and a fragmentation profile. As shown, a nucleosome dynamics profile may contain information derived from any one or more of: (a) nucleosome positional information; (b) nucleosome occupancy; or (c) nucleosome fuzziness. Feature identification and assessment may be performed using a combined prediction model 108 using the information obtained from a single assay (i.e., the single non-disruptive methylation sequencing technique performed on a cfDNA sample) to determine an epigenetic signature 110. In some embodiments, the workflow 100 is configured for the discovery of a multimodal epigenetic signature. In some embodiments, the workflow 100 is configured for the assessment of a multimodal epigenetic signature in a sample from an individual, such as for the diagnosis of a disease, e.g., a cancer.

The multimodal epigenetic signatures taught herein provide insightful information regarding the biological state of an individual, such as a disease state and/or response to treatment, and may be used for a diverse array of methods. In certain aspects, provided herein is a method of determining an epigenetic signature. In other aspects, provided herein is a method of generating an epigenetic signature using a machine learning model. In other aspects, provided herein is a method of diagnosing a disease in an individual using an epigenetic signature. In other aspects, provided herein is a method of treating a disease in an individual comprising diagnosing the disease in the individual using an epigenetic signature. In certain aspects, provided herein is a method of identifying a disease epigenetic signature in an individual comprising training a machine learning model to identify the disease epigenetic signature.

The multimodal epigenetic signatures provided herein may comprise information obtained from any combination of two or more of methylation profile, a nucleosome dynamics profile, and a fragmentation profile. As described herein, the methylation profile comprises information derived from one or more methylation sites. The nucleosome dynamics profile comprises information derived from any one or more of: (a) nucleosome positional information; (b) nucleosome occupancy; or (c) nucleosome fuzziness. The fragmentation profile comprises information derived from read distributions in one or more base length windows. In some embodiments, the epigenetic signature comprises features from the methylation profile and the nucleosome dynamics profile (including features from any of, or combination of, nucleosome positional information, nucleosome occupancy, or nucleosome fuzziness). In some embodiments, the epigenetic signature comprises features from the methylation profile and the fragmentation profile. In some embodiments, the epigenetic signature comprises features from the nucleosome dynamics profile (including features from any of, or combination of, nucleosome positional information, nucleosome occupancy, or nucleosome fuzziness) and the fragmentation profile. In some embodiments, the epigenetic signature comprises features from the methylation profile, the nucleosome dynamics profile (including features from any of, or combination of, nucleosome positional information, nucleosome occupancy, or nucleosome fuzziness), and the fragmentation profile. In some embodiments, the nucleosome dynamics profile comprises information derived from nucleosome positional information. In some embodiments, the nucleosome dynamics profile comprises information derived from nucleosome occupancy. In some embodiments, the nucleosome dynamics profile comprises information derived from nucleosome fuzziness. In some embodiments, the epigenetic signature is indicative of whether the individual has a disease.

In the following sections, additional description of the various aspects of the epigenetic signatures and associated methods taught herein are provided. Such description in a modular fashion is not intended to limit the scope of the disclosure, and based on the teachings provided herein one of ordinary skill in the art will readily appreciate that certain modules can be integrated, at least in part. The section heading used herein are for organizational purposes only and are not to be construed as limiting the subject matter described.

### I. Non-disruptive Methylation Sequencing Techniques

In certain aspects, the methods provided herein involve non-disruptive methylation sequencing techniques, and/or use of data obtained therefrom. In some embodiments, the non-disruptive methylation sequencing technique is configured to produce sequencing information, such as sequencing reads, suitable for use in determining one or more of a methylation profile, a nucleosome dynamics profile, or a fragmentation profile from a single assay. In some embodiments, the non-disruptive methylation sequencing technique comprises use of an enzyme to convert a nucleic acid base such that it can be distinguished from sequencing information, such as via deamination of an unmethylated cytosine to a uracil.

In some embodiments, the method provided herein further comprises performing the non-disruptive methylation sequencing technique. In some embodiments, the non-disruptive methylation sequencing technique is an enzymatic methyl-seq (EM-seq) technique. In some embodiments, the non-disruptive methylation sequencing technique comprises: (a) enzymatically modifying methylated cytosines (such as 5-methylcytosine (5 mc) and 5-hydroxymethylcytosine (5 hmC)) to prevent deamination in further enzymatic steps; (b) enzymatically converting unmethylated cytosines to uracils; (c) performing PCR amplification (thereby converting uracils to thymines; and (d) sequencing using a next generation sequencing technique. Various techniques for performing a non-disruptive methylation sequencing technique have been described in the art. See, e.g., Vaisvila et al., Genome Res, 31, 2021, which is incorporated herein in its entirety. In some embodiments, enzymatically modifying methylated cytosines is performed using TET2 and/or T4-BGT. In some embodiments, the non-disruptive methylation sequencing technique comprises enzymatically converting unmethylated cytosines to uracil using APOBEC3A. In some embodiments, the non-disruptive methylation sequencing technique comprises subjecting a sample comprising genomic DNA, such as a cfDNA sample, to a next generation sequencing library preparation technique. In some embodiments, the next generation sequencing library preparation technique comprises shearing the genomic DNA, such as to obtain a DNA size of less than about 500 base pairs, such as less than about any of 450 base pairs, 400 base pairs, 350 base pairs, or 300 base pairs. In some embodiments, the next generation sequencing library preparation technique comprises a step of end prep of sheared DNA. In some embodiments, the next generation sequencing library preparation technique comprises a step of adaptor ligation. In some embodiments, the next generation sequencing library preparation technique comprises a step of cleaning up adaptor ligated DNA. In some embodiments, the cleaned and ligated DNA is subjected to oxidative enzymes, such as TET2 and/or T4-BGT, to modify methylated cytosines (5-methylcytosines and 5-hydroxymethylcytosines). In some embodiments, the next generation sequencing library preparation technique comprises a step of cleaning enzyme oxidized DNA. In some embodiments, the oxidized DNA is further subjected to enzymatic cytosine deamination (such as using APOBEC3A). In some embodiments, the next generation sequencing library preparation technique comprises a step of PCR amplification of the deaminated DNA. In some embodiments, the next generation sequencing library preparation technique comprises a step of sequencing and quantification. In some embodiments, the method comprises adding a control to the sample comprising genomic DNA, e.g., prior to performing any enzymatic conversion steps.

In some embodiments, the non-disruptive methylation sequencing technique is performed based on targeted genetic locations. In some embodiments, the non-disruptive methylation sequencing technique is performed across a whole genome.

In some embodiments, the data obtained from the non-disruptive methylation sequencing technique comprises a plurality of sequence reads. In some embodiments, the non-disruptive methylation sequencing technique is performed to a sequencing depth of about 50x to about 500x. In some embodiments, the non-disruptive methylation sequencing technique is performed to a sequencing depth of at least about 50x, such as at least about any of 75x, 100x, 125x, 150x, 175x, 200x, 225x, 250x, 275x, 300x, 325x, 350x, 375x, 400x, 425x, 450x, 475x, or 500x. In some embodiments, the non-disruptive methylation sequencing technique is performed to a sequencing depth of about any of 50x, 75x, 100x, 125x, 150x, 175x, 200x, 225x, 250x, 275x, 300x, 325x, 350x, 375x, 400x, 425x, 450x, 475x, or 500x.

In some embodiments, the method further comprises processing the plurality of sequence reads to remove low-quality reads and/or remove adaptor contamination and/or filter based on sequence read size. In some embodiments, the method further comprises aligning the plurality of sequence reads with a reference genome.

In some embodiments, the methods provided herein involve non-disruptive methylation sequencing techniques in combination with one or more additional sequencing techniques. In some embodiments, the one or more additional sequencing techniques comprise next-generation sequencing, such as deep sequencing, droplet digital PCR, and/or pyrosequencing. In some embodiments, the sequencing investigates DNA mutations (e.g., cfDNA mutations), RNA, micoRNA, or any combination thereof. For example, the method may comprise performing the non-disruptive methylation sequencing and deep sequencing (e.g., to evaluate mutations). In some embodiments, the method comprises performing non-disruptive methylation sequencing to obtain a methylation profile comprising information derived from one or more methylation sites; and performing another sequencing technique (e.g., deep sequencing) to obtain a nucleosome dynamics profile comprising information derived from any one or more of: (a) nucleosome positional information; (b) nucleosome occupancy; or (c) nucleosome fuzziness. In some embodiments, the method comprises performing non-disruptive methylation sequencing to obtain a methylation profile comprising information derived from one or more methylation sites; and performing one or more additional sequencing technique (e.g., deep sequencing) to obtain a fragmentation profile comprising information derived from read distributions in one or more base length windows. In some embodiments, the method comprises performing non-disruptive methylation sequencing to obtain a methylation profile comprising information derived from one or more methylation sites; and performing one or more additional sequencing technique (e.g., deep sequencing) to obtain a nucleosome dynamics profile comprising information derived from any one or more of: (a) nucleosome positional information; (b) nucleosome occupancy; or (c) nucleosome fuzziness; and performing another sequencing technique (e.g., deep sequencing) to obtain a fragmentation profile comprising information derived from read distributions in one or more base length windows.

Suitable sequencing techniques useful for non-disruptive methylation sequencing techniques described herein are well known in the art. In some embodiments, such sequencing techniques involve (i) amplification and detection, or (ii) direct detection, by a variety of methods such as (a) PCR (sequence-specific amplification) such as TaqMan(R), (b) DNA sequencing of untreated and treated DNA, (c) sequencing by ligation of dye-modified probes (including cyclic ligation and cleavage), (d) pyrosequencing, (e) single-molecule sequencing, (f) mass spectroscopy, or (g) Southern blot analysis.

In some embodiments, restriction enzyme digestion of PCR products amplified from enzymatically-converted DNA may be used, e.g., the method described by Sadri and Hornsby (Sadri et al., 1996, Nucl. Acids Res. 24:5058-5059), or COBRA (Combined Bisulfite Restriction Analysis) (Xiong and Laird, 1997, Nucleic Acids Res. 25:2532-2534). COBRA analysis is a quantitative methylation assay useful for determining DNA methylation levels at specific gene loci in small amounts of genomic DNA. Briefly, restriction enzyme digestion is used to reveal methylation-dependent sequence differences in PCR products of enzymatically-converted DNA. PCR amplification of the converted DNA is then performed using primers specific for the CpG sites of interest, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels.

In some embodiments, the methylation profile of selected CpG sites is determined using methylation-Specific PCR (MSP). MSP allows for assessing the methylation status of virtually any group of CpG sites within a CpG island, independent of the use of methylation-sensitive restriction enzymes (Herman et al., 1996, Proc. Nat. Acad. Sci. USA, 93, 9821-9826; U.S. Pat. Nos. 5,786,146, 6,017,704, 6,200,756, 6,265,171 (Herman and Baylin); U.S. Pat. Pub. No. 2010/0144836 (Van England et al.); which are hereby incorporated by reference in their entirety). Briefly, DNA is enzymatically deaminated to convert unmethylated, but not methylated cytosines to uracil, and subsequently amplified with primers specific for methylated versus unmethylated DNA. In some instances, typical reagents (e.g., as might be found in a typical MSP-based kit) for MSP analysis include, but are not limited to: methylated and unmethylated PCR primers for specific gene (or methylation-altered DNA sequence or CpG island), optimized PCR buffers and deoxynucleotides, and specific probes. One may use quantitative multiplexed methylation specific PCR (QM-PCR), as described by Fackler et al., 2004, Cancer Res. 64(13) 4442-4452; or, Fackler et al., 2006, Clin. Cancer Res. 12(11 Pt 1) 3306-3310.

In some embodiments, the non-disruptive methylation sequencing technique comprises MethyLight and/or Heavy Methyl Methods. The MethyLight and Heavy Methyl assays are a high-throughput quantitative methylation assay that utilizes fluorescence-based real-time PCR (Taq Man(R)) technology that requires no further manipulations after the PCR step (Eads, C. A. et al., 2000, Nucleic Acid Res. 28, e 32; Cottrell et al., 2007, J. Urology 177, 1753, U.S. Pat. No. 6,331,393 (Laird et al.), the contents of which are hereby incorporated by reference in their entirety).

In some embodiments, the non-disruptive methylation sequencing technique comprises Ms-SNuPE techniques. The Ms-SNuPE technique is a quantitative method for assessing methylation differences at specific CpG sites based on enzymatic deamination of DNA, followed by single-nucleotide primer extension (Gonzalgo and Jones, 1997, Nucleic Acids Res. 25, 2529-2531).

In some embodiments, provided are methods for quantifying the average methylation density in a target sequence within a population of genomic DNA. In some instances, quantitative amplification methods (e.g., quantitative PCR or quantitative linear amplification) are used. Methods of quantitative amplification are disclosed in, e.g., U.S. Pat. Nos. 6,180,349; 6,033,854; and 5,972,602, as well as in, e.g., DeGraves, et al., 34(1) Biotechniques 106-15 (2003); Deiman B, et al., 20(2) Mol. Biotechnol. 163-79 (2002); and Gibson et al., 6 Genome Res. 995-1001 (1996).

In some embodiments, the methods provided herein comprise a sequence-based analysis. For example, once it is determined that one particular genomic sequence from a sample is hypermethylated or hypomethylated compared to its counterpart, the amount of this genomic sequence can be determined. Subsequently, this amount can be compared to a standard control value and used to determine the present of liver cancer in the sample. In many instances, it is desirable to amplify a nucleic acid sequence using any of several nucleic acid amplification procedures which are well known in the art. Specifically, nucleic acid amplification is the chemical or enzymatic synthesis of nucleic acid copies which contain a sequence that is complementary to a nucleic acid sequence being amplified (template). The methods and kits may use any nucleic acid amplification or detection methods known to one skilled in the art, such as those described in U.S. Pat. No. 5,525,462 (Takarada et al.); U.S. Pat. No. 6,114,117 (Hepp et al.); U.S. Pat. No. 6,127,120 (Graham et al.); U.S. Pat. No. 6,344,317 (Urnovitz); U.S. Pat. No. 6,448,001 (Oku); U.S. Pat. No. 6,528,632 (Catanzariti et al.); and PCT Pub. No. WO 2005/111209 (Nakajima et al.); all of which are incorporated herein by reference in their entirety.

In some embodiments, the nucleic acids are amplified by PCR amplification using methodologies known to one skilled in the art. One skilled in the art will recognize, however, that amplification can be accomplished by any known method, such as ligase chain reaction (LCR), Q-replicas amplification, rolling circle amplification, transcription amplification, self-sustained sequence replication, nucleic acid sequence-based amplification (NASBA), each of which provides sufficient amplification. Branched-DNA technology is also optionally used to qualitatively demonstrate the presence of a sequence of the technology, which represents a particular methylation pattern, or to quantitatively determine the amount of this particular genomic sequence in a sample. Nolte reviews branched-DNA signal amplification for direct quantitation of nucleic acid sequences in clinical samples (Nolte, 1998, Adv. Clin. Chem. 33:201-235).

The PCR process is well known in the art and include, for example, reverse transcription PCR, ligation mediated PCR, digital PCR (dPCR), or droplet digital PCR (ddPCR). For a review of PCR methods and protocols, see, e.g., Innis et al., eds., PCR Protocols, A Guide to Methods and Application, Academic Press, Inc., San Diego, Calif. 1990; U.S. Pat. No. 4,683,202 (Mullis). PCR reagents and protocols are also available from commercial vendors, such as Roche Molecular Systems. In some instances, PCR is carried out as an automated process with a thermostable enzyme. In this process, the temperature of the reaction mixture is cycled through a denaturing region, a primer annealing region, and an extension reaction region automatically. Machines specifically adapted for this purpose are commercially available.

Suitable next generation sequencing technologies are widely available. Examples include the 454 Life Sciences platform (Roche, Branford, CT) (Margulies et al., 2005 Nature, 437, 376-380); Illumina's Genome Analyzer, GoldenGate Methylation Assay, or Infinium Methylation Assays, i.e., Infinium HumanMethylation 27K BeadArray or VeraCode GoldenGate methylation array (Illumina, San Diego, CA; Bibkova et al., 2006, Genome Res. 16, 383-393; U.S. Pat. Nos. 6,306,597 and 7,598,035 (Macevicz); 7,232,656 (Balasubramanian et al.)); QX200™ Droplet Digital™ PCR System from Bio-Rad; or DNA Sequencing by Ligation, SOLiD System (Applied Biosystems/Life Technologies; U.S. Pat. Nos. 6,797,470, 7,083,917, 7,166,434, 7,320,865, 7,332,285, 7,364,858, and 7,429,453 (Barany et al.); the Helicos True Single Molecule DNA sequencing technology (Harris et al., 2008 Science, 320, 106-109; U.S. Pat. Nos. 7,037,687 and 7,645,596 (Williams et al.); U.S. Pat. No. 7, 169,560 (Lapidus et al.); U.S. Pat. No. 7,769,400 (Harris)), the single molecule, real-time (SMRT™) technology of Pacific Biosciences, and sequencing (Soni and Meller, 2007, Clin. Chem. 53, 1996-2001); semiconductor sequencing (Ion Torrent; Personal Genome Machine); DNA nanoball sequencing; sequencing using technology from Dover Systems (Polonator), and technologies that do not require amplification or otherwise transform native DNA prior to sequencing (e.g., Pacific Biosciences and Helicos), such as nanopore-based strategies (e.g., Oxford Nanopore, Genia Technologies, and Nabsys). These systems allow the sequencing of many nucleic acid molecules isolated from a specimen at high orders of multiplexing in a parallel fashion. Each of these platforms allows sequencing of clonally expanded or non-amplified single molecules of nucleic acid fragments. Certain platforms involve, for example, (i) sequencing by ligation of dye-modified probes (including cyclic ligation and cleavage), (ii) pyrosequencing, and (iii) single-molecule sequencing.

In some embodiments, the analyzing described above comprises quantitatively detecting the methylation status of the amplified product. In some cases, the detection comprises a real-time quantitative probe-based PCR or a digital probe-based PCR. In some cases, the detection comprises a real-time quantitative probe-based PCR. In other cases, the detection comprises a digital probe-based PCR, optionally, a digital droplet PCR.

### II. Methylation Profiles

In certain aspects, the methods provided herein comprise a multimodal epigenetic signature comprising one or more features obtained from a methylation profile. As described herein, methylation profiles are based on the presence and/or absence of methylation at one or more methylation sites. In some embodiments, the methylation profile comprises a qualitative feature of one or more methylation sites, such as presence or absence of methylation at a methylation site. In some embodiments, the methylation profile comprises a quantification feature of one or more methylation sites, such as obtained via a beta value and/or Cellular Heterogeneity-Adjusted cLonal Methylation (CHALM).

DNA methylation is the attachment of a methyl group at the CS-position of the nucleotide base cytosine and the N6-position of adenine. Methylation of adenine primarily occurs in prokaryotes, while methylation of cytosine occurs in both prokaryotes and eukaryotes. In some embodiments, methylation of cytosine occurs in the CpG dinucleotides motif. In some embodiments, cytosine methylation occurs in, for example CHG and CHH motifs, where H is adenine, cytosine or thymine. In some embodiments, one or more CpG dinucleotide motif or CpG site forms a CpG island, a short DNA sequence rich in CpG dinucleotide. In some embodiments, a CpG island is present in the 5′ region of about one half of all human genes. CpG islands are typically, but not always, between about 0.2 to about 1 kb in length. Cytosine methylation further comprises 5-methylcytosine (5-mCyt) and 5-hydroxymethylcytosine. The CpG (cytosine-phosphate-guanine) or CG motif refers to regions of a DNA molecule where a cytosine nucleotide occurs next to a guanine nucleotide in the linear strand. In some embodiments, a cytosine in a CpG dinucleotide is methylated to form 5-methylcytosine. In some embodiments, a cytosine in a CpG dinucleotide is methylated to form 5-hydroxymethylcytosine.

In some embodiments, one or more DNA regions, such as a methylation site, are hypermethylated. In such cases, hypermethylation refers to an increase in methylation event of a region relative to a reference region (such as another region of DNA or the same region in a control sample). In some cases, hypermethylation is observed in one or more cancer types, and is useful, for example, as a diagnostic marker and/or a prognostic marker. In some embodiments, one or more DNA regions are hypomethylated. In some embodiments, hypomethylation refers to a loss of the methyl group in the 5-methylcytosine nucleotide in a first region relative to a reference region (such as another region of DNA or the same region in a control sample). In some embodiments, hypomethylation is observed in one or cancer types, and is useful, for example, as a diagnostic marker and/or a prognostic marker.

In some embodiments, as discussed herein, methylation at one or more methylation sites is assessed using a non-disruptive methylation sequencing technique, such as EM-seq. In some embodiments, the methylation assessment methods encompassed herein comprise use of a probe to assess methylation at a methylation site. In some embodiments, the methylation assessment methods encompassed herein comprise use of a panel of probes to assess methylation at a plurality of methylation sites.

In some embodiments, the one or more methylation sites of the methylation profile are from any of those provided in the Illumina Infinium HumanMethylation450 BeadChip (450K) available at the time of filing the instant application. The methylation markers included in the Illumina Infinium HumanMethylation450 BeadChip (450K) are known, see, e.g., Wang et al., BMC Bioinformatics, 19, 2018, which is incorporated herein by reference in its entirety. In some embodiments, the one or more methylation sites of the methylation profile are from any of those provided in the Twist Methylome panel available at the time of filing the instant application.

In some embodiments, the one or more methylation sites of the methylation profile are from selected CpG methylation sites. In some embodiments, the one or more methylation sites of the methylation profile are from more than, at least, or about 1, 5, 10, 15, 20, 25, 30, 35, 40, 50, 75, or 100, 150, 200, 250, 300, 400, 500, 750, 1000, 2000, 2500, 3000, 4000, 5000, 7500, 10000, 20000, 25000, 30000, 40000, 50000, 75000, 100000, 200000, 300000, 400000, 500000, 600000 and 700000 selected CpG methylation sites. In some embodiments, the one or more methylation sites of the methylation profile are from about 1 to about 500,000 selection CpG methylation sites. In some embodiments, the one or more methylation sites of the methylation profile are cg18081940, cg23089825, cg16395183, cg19811148, cg07790615, cg20996351, cg04977528, cg24465685, cg20428713, cg13678973, cg25339566, cg16596317, cg23786625, cg11328303, cg19578660, cg02272851, cg10298052, cg13585930, cg23575688, cg12394201, cg08149193, cg18854419, cg07603330, cg10658542, cg13099890, cg22302985, cg13596497, cg14507533, cg25366582, cg22396555, cg10566012, cg05168229, cg10795666, cg25078444, cg16038120, cg23883632, cg18380808, cg13615592, cg00250422, cg19691260, cg16558770, cg15681853, cg03397724, cg10514097, cg06674117, cg16047279, cg12127472, cg08843809, cg08697732, cg06384763, cg04203646, cg17112426, cg08278741, cg14587524, cg26087117, cg18320766, cg08063125, cg10004780, cg18921980, cg02514318, cg20002504, cg18897632, cg15313459, cg19370054, cg16564824, cg02631468, cg01471196, cg23770904, cg18412834, cg24080247, cg11549874, cg13155421, cg19442495, cg22536150, cg05413061, cg23346462, cg09477895, cg13605674, cg13314965, cg09417547, cg00181669, cg23967169, cg10237419, cg21077559, cg27600205, cg19755714, cg18797590, cg00699993, cg06485940, cg27661394, cg00939495, cg11036833, cg23915769, cg07224726, cg02022733, cg03640756, cg15361590, cg04598517, cg06782035, cg13954457, cg25482900, cg20952257, cg14062050, cg01881524, cg11538641, cg11387340, cg05389236, cg19419054, cg10575547, cg17240815, cg24772267, cg00920327, cg00772257, cg26253500, cg23244488, cg22778435, cg26065247, cg02088996, cg19868631, cg22280038, cg07803375, cg20230721, cg03333330, cg21517947, cg10406295, cg05166490, cg07739205, cg20980783, cg06617456, cg01568998, cg13407456, cg23758305, cg20675505, cg07585876, cg03734437, cg13410764, or any combination thereof.

In some embodiments, the one or more methylation sites of the methylation profile are cg18081940, cg23089825, cg16395183, cg19811148, cg07790615, cg20996351, cg04977528, cg24465685, cg20428713, cg13678973, cg25339566, cg16596317, cg23786625, cg11328303, cg19578660, cg02272851, cg10298052, cg13585930, cg23575688, cg12394201, cg08149193, cg18854419, cg07603330, cg10658542, cg13099890, cg22302985, cg13596497, cg14507533, cg25366582, cg22396555, cg10566012, cg05168229, cg10795666, cg25078444, cg16038120, cg23883632, cg18380808, cg13615592, cg00250422, cg19691260, cg16558770, cg15681853, cg03397724, cg10514097, cg06674117, cg16047279, cg12127472, cg08843809, cg08697732, cg06384763, cg04203646, cg17112426, cg08278741, cg14587524, cg26087117, cg18320766, cg08063125, cg10004780, cg18921980, cg02514318, cg20002504, cg18897632, cg15313459, cg19370054, cg16564824, cg02631468, cg01471196, cg23770904, cg18412834, cg24080247, cg11549874, cg13155421, cg19442495, cg22536150, cg05413061, cg23346462, cg09477895, cg13605674, cg13314965, cg09417547, cg00181669, cg23967169, cg10237419, cg21077559, cg27600205, cg19755714, cg18797590, cg00699993, cg06485940, cg27661394, cg00939495, cg11036833, cg23915769, cg07224726, cg02022733, cg03640756, cg15361590, cg04598517, cg06782035, cg13954457, cg25482900, cg20952257, cg14062050, cg01881524, cg11538641, cg11387340, cg05389236, cg19419054, cg10575547, cg17240815, cg24772267, cg00920327, cg00772257, cg26253500, cg23244488, cg22778435, cg26065247, cg02088996, cg19868631, cg22280038, cg07803375, cg20230721, cg03333330, cg21517947, cg10406295, cg05166490, cg07739205, cg20980783, cg06617456, cg01568998, cg13407456, cg23758305, cg20675505, cg07585876, cg03734437, and cg13410764.

In some embodiments, the one or more methylation sites of the methylation profile comprise one or more gene promoter region methylation sites.

In some embodiments, the methylation profile comprises quantitative information from at least one of the one or more methylation sites.

In some embodiments, the quantitative information is based on a (3-value from the at least one methylation sites. In some embodiments, the methylation profile comprises a quantitative value threshold, such as to indicate when the level of methylation at a methylation site has satisfied a condition, such as hypermethylation above a certain beta value or hypomethylation below a certain beta value.

In some embodiments, the quantitative information is based on a Cellular Heterogeneity-Adjusted cLonal Methylation (CHALM) ratio from the at least one methylation sites. See, e.g., Xu et al., Nat Commun, 12, 2021. CHALM is a method for quantifying cell heterogeneity-adjusted mean methylation. CHALM quantifies the promoter methylation as the ratio of methylated reads (with ≥1 mCpG) to total reads mapped to a given promoter region.

In certain embodiments, the information obtained from the methylation sites is mathematically combined and the combined value is correlated to the underlying therapeutic question, such as a diagnostic question. In some embodiments, information from a plurality of methylation sites is combined by any appropriate state of the art mathematical method. Well-known mathematical methods for correlating a biomarker combination to, e.g., a disease status employ methods like discriminant analysis (DA) (e.g., linear-, quadratic-, regularized-DA), Discriminant Functional Analysis (DFA), Kernel Methods (e.g., SVM), Multidimensional Scaling (MDS), Nonparametric Methods (e.g., k-Nearest-Neighbor Classifiers), PLS (Partial Least Squares), Tree-Based Methods (e.g., Logic Regression, CART, Random Forest Methods, Boosting/Bagging Methods), Generalized Linear Models (e.g., Logistic Regression), Principal Components based Methods (e.g., SIMCA), Generalized Additive Models, Fuzzy Logic based Methods, Neural Networks and Genetic Algorithms based Methods. In some embodiments, the mathematical model comprises a p-value test or t-value test or F-test. Rated (best first, i.e., low p- or t-value) methylation sites may then be subsequently selected and added to the methylation panel until a certain value is reached, such as a diagnostic value with a desired confidence level. Such methods include a random-variance t-test (Wright G. W. and Simon R, Bioinformatics 19:2448-2455, 2003). The skilled artisan will have no problem in selecting an appropriate method to evaluate methylation sites and combinations described herein. Details relating to these statistical methods are found in the following references: Ruczinski et al., 12 J. of Computational and Graphical Statistics 475-511 (2003); Friedman, J. H., 84 J. of the American Statistical Association 165-75 (1989); Hastie, Trevor, Tibshirani, Robert, Friedman, Jerome, The Elements of Statistical Learning, Springer Series in Statistics (2001); Breiman, L., Friedman, J. H., Olshen, R. A., Stone, C. J. Classification and regression trees, California: Wadsworth (1984); Breiman, L., 45 Machine Learning 5-32 (2001); Pepe, M. S., The Statistical Evaluation of Medical Tests for Classification and Prediction, Oxford Statistical Science Series, 28 (2003); and Duda, R. 0., Hart, P. E., Stork, D. O., Pattern Classification, Wiley Interscience, 2nd Edition (2001).

In some embodiments, the methods provided herein include models for prediction. These models may be based on the Compound Covariate Predictor (Radmacher et al., J of Computational Biology 9:505-511, 2002), Diagonal Linear Discriminant Analysis (Dudoit et al., Journal of the American Statistical Association 97:77-87, 2002), Nearest Neighbor Classification (also Dudoit et al.), and Support Vector Machines with linear kernel (Ramaswamy et al., PNAS USA 98:15149-54, 2001). Another classification method is the greedy-pairs method described by Bo and Jonassen (Genome Biology 3(4):research0017.1-0017.11, 2002). The greedy-pairs approach starts with ranking all markers based on their individual t-scores on the training set. This method attempts to select pairs of markers that work well together to discriminate the classes. Furthermore, a binary tree classifier for utilizing a methylation profile is optionally used to predict the class of future samples. The first node of the tree incorporated a binary classifier that distinguished two subsets of the total set of classes. The individual binary classifiers are based on the “Support Vector Machines” incorporating markers that were differentially expressed among markers at the significance level (e.g., 0.01, 0.05 or 0.1) as assessed by the random variance t-test (Wright G. W. and Simon R. Bioinformatics 19:2448-2455, 2003). Classifiers for all possible binary partitions are evaluated and the partition selected is that for which the cross-validated prediction error is minimum. The process is then repeated successively for the two subsets of classes determined by the previous binary split. The prediction error of the binary tree classifier can be estimated by cross-validating the entire tree building process. This overall cross-validation includes re-selection of the optimal partitions at each node and re-selection of the markers used for each cross-validated training set as described by Simon et al. (Simon et al., Journal of the National Cancer Institute 95:14-18, 2003). Several-fold cross validation in which a fraction of the samples is withheld, a binary tree developed on the remaining samples, and then class membership is predicted for the samples withheld. This is repeated several times, each time withholding a different percentage of the samples. The samples are randomly partitioned into fractional test sets (Simon R and Lam A. BRB-ArrayTools User Guide, version 3.2. Biometric Research Branch, National Cancer Institute).

### III. Nucleosome Dynamics Profiles

In certain aspects, the methods provided herein comprise a multimodal epigenetic signature comprising one or more features obtained from a nucleosome dynamics profile. As described herein, nucleosome dynamics profiles include location-based information of one or more nucleosomes that can be ascertained from sequencing data, such as obtained from a cfDNA sample. For example, in some embodiments, the nucleosome dynamics profile comprises information based on the presence and/or absence of a nucleosome at a locus on genomic DNA, including genomic DNA in systemic circulation as cfDNA.

In some embodiments, the nucleosome dynamics profile comprises nucleosome positional information, e.g., as represented by a window protection score (WPS). WPS is determined via analysis of the number of sequenced DNA fragments completely spanning a window, e.g., 120 bp window, centered at a given genomic coordinate, minus the number of fragments with an endpoint within that same window, and correlates with the location of a nucleosome. In some embodiments, the nucleosome positional information is based on a WPS. In some embodiments, the WPS is an average WPS. Methods for determining WPS are known in the art, see, e.g., Snyder et al., Cell, 164, 2016, which is incorporated herein by references in its entirety.

In some embodiments, the nucleosome dynamics profile comprises nucleosome occupancy information. Nucleosome occupancy reflects the frequency at which a nucleosome occupies a nucleosome position. In some embodiments, the nucleosome occupancy is based on the frequency a nucleosome occupies a genomic region. In some embodiments, sliding windows in target regions are used to assess nucleosome occupancy, e.g., windows of 250-2000 base pairs that slide in 10 base pair steps across a target region. In some embodiments, the nucleosome occupancy is obtained via normalized read coverage measured by counts per million. Tools for determining normalized read coverage are known in the art, including bamCoverage from deepTools.

In some embodiments, the nucleosome dynamics profile comprises nucleosome fuzziness information. Nucleosome fuzziness reflects the deviation of measured nucleosome positions. In some embodiments, the nucleosome fuzziness is based on the deviation of a nucleosome position from a prefer nucleosome position.

In some embodiments, the nucleosome dynamics profile comprises information derived from nucleosome positional information and nucleosome occupancy. In some embodiments, the nucleosome dynamics profile comprises information derived from nucleosome positional information and nucleosome fuzziness. In some embodiments, the nucleosome dynamics profile comprises information derived from nucleosome occupancy and nucleosome fuzziness. In some embodiments, the nucleosome dynamics profile comprises information derived from nucleosome positional information, nucleosome occupancy, and nucleosome fuzziness.

In some embodiments, the nucleosome dynamic information is obtained via dynamic analysis of nucleosome position and occupancy by sequencing (DANPOS). See, e.g., Chen et al., Genome Res, 23, 2013, which is incorporated herein by reference in its entirety. DANPOS, and further versions thereof such as DANPOS 2, is a comprehensive bioinformatics pipeline designed for dynamic nucleosome analysis at single-nucleotide resolution. In some embodiments, the nucleosome dynamics profile comprises a locus-specific nucleosome score based on any one or more of: (a) nucleosome positional information; (b) nucleosome occupancy; or (c) nucleosome fuzziness.

### IV. Fragmentation Profiles

In certain aspects, the methods provided herein comprise a multimodal epigenetic signature comprising one or more features obtained from a fragmentation profile. As described herein, fragmentation profiles are based on the fraction of nucleic acid fragments in one or more nucleic acid base length windows. As described in more detail below, the fraction of nucleic acid fragments may be based on a desired population of nucleic acid fragments, such as all sequencing reads from an assay or a subset thereof. In some embodiments, the population of nucleic acid fragments used to assess a fragmentation profile comprises fragments associated with a targeted location, such as a targeted chromosome or one or more loci.

In some embodiments, the fragmentation profile comprises one or more nucleic acid base length windows occupying the range of about 30 bases in length to about 250 bases in length, such as any of about 60 bases in length to about 200 bases in length, about 80 bases in length to about 200 bases in length, about 120 bases in length to about 220 bases in length, about 120 bases in length to about 180 bases in length, or about 140 bases in length to about 200 bases in length. In some embodiments, the fragmentation profile comprises one or more nucleic acid base length windows occupying nucleic acid have a base length of about 250 bases or less, such as about any of 240 bases or less, 230 bases or less, 220 bases or less, 210 bases or less, 200 bases or less, 190 bases or less, 180 bases or less, 170 bases or less, 160 bases or less, 150 bases or less, 140 bases or less, 130 bases or less, or 120 bases or less. In some embodiments, the fragmentation profile comprises one or more nucleic acid base length windows encompassing a nucleic acid base length of about 147 bases in length and/or about 167 bases in length.

In some embodiments, the fragmentation profile comprises one or more nucleic acid base length windows from: about 80 bases in length to about 150 bases in length, about 80 bases in length to about 155 bases in length, about 80 bases in length to about 160 bases in length, about 80 bases in length to about 165 bases in length, about 125 bases in length to about 155 bases in length, about 170 bases in length to about 175 bases in length, about 170 bases in length to about 200 bases in length, about 175 bases in length to about 200 bases in length, about 151 bases in length to about 200 bases in length, about 156 bases in length to about 200 bases in length, about 161 bases in length to about 200 bases in length, or about 166 bases in length to about bases in length. In some embodiments, the fragmentation profile comprises nucleic acid base length windows of about 80 bases in length to about 150 bases in length, about 80 bases in length to about 155 bases in length, about 80 bases in length to about 160 bases in length, about 80 bases in length to about 165 bases in length, about 125 bases in length to about 155 bases in length, about 170 bases in length to about 175 bases in length, about 170 bases in length to about 200 bases in length, and about 175 bases in length to about 200 bases in length.

In some embodiments, the fragmentation profile comprises one or more ratios of a first nucleic base length window over a second nucleic base length window. In some embodiments, the ratio is one or more of about 80 bases in length to about bases in length 150 bases in length over about 151 bases in length to about 200 bases in length, about 80 bases in length to about bases in length 155 bases in length over about 156 bases in length to about 200 bases in length, about 80 bases in length to about bases in length 160 bases in length over about 161 bases in length to about 200 bases in length, or about 80 bases in length to about bases in length 165 bases in length over about 166 bases in length to about 200 bases in length. In some embodiments, the fragmentation profile comprises ratios of about 80 bases in length to about bases in length 150 bases in length over about 151 bases in length to about 200 bases in length, about 80 bases in length to about bases in length 155 bases in length over about 156 bases in length to about 200 bases in length, about 80 bases in length to about bases in length 160 bases in length over about 161 bases in length to about 200 bases in length, and about 80 bases in length to about bases in length 165 bases in length over about 166 bases in length to about 200 bases in length.

In some embodiments, the nucleic base length window is about 5 bases in length to about 150 bases in length, such as any of about 10 bases in length to about 120 bases in length, about 40 bases in length to about 100 bases in length, or about 60 bases in length to about 80 bases in length. In some embodiments, the nucleic base length window is at least about 5 bases in length, such as at least about any of 10 bases in length, 15 bases in length, 20 bases in length, 25 bases in length, 30 bases in length, 35 bases in length, 40 bases in length, 45 bases in length, 50 bases in length, 55 bases in length, 60 bases in length, 65 bases in length, 70 bases in length, 75 bases in length, 80 bases in length, 85 bases in length, 90 bases in length, 95 bases in length, 100 bases in length, 105 bases in length, 110 bases in length, 115 bases in length, 120 bases in length, 125 bases in length, 130 bases in length, 135 bases in length, 140 bases in length, 145 bases in length, or 150 bases in length. In some embodiments, the nucleic base length window about 150 or fewer bases in length, such as about any of 145 or fewer bases in length, 140 or fewer bases in length, 135 or fewer bases in length, 130 or fewer bases in length, 125 or fewer bases in length, 120 or fewer bases in length, 115 or fewer bases in length, 110 or fewer bases in length, 105 or fewer bases in length, 100 or fewer bases in length, 95 or fewer bases in length, 90 or fewer bases in length, 85 or fewer bases in length, 80 or fewer bases in length, 75 or fewer bases in length, 70 or fewer bases in length, 65 or fewer bases in length, 60 or fewer bases in length, 55 or fewer bases in length, 50 or fewer bases in length, 45 or fewer bases in length, 40 or fewer bases in length, 35 or fewer bases in length, 30 or fewer bases in length, 25 or fewer bases in length, 20 or fewer bases in length, 15 or fewer bases in length, or 10 or fewer bases in length. In some embodiments, the nucleic base length window is about any of 10 bases in length, 15 bases in length, 20 bases in length, 25 bases in length, 30 bases in length, 35 bases in length, 40 bases in length, 45 bases in length, 50 bases in length, 55 bases in length, 65 bases in length, 70 bases in length, 75 bases in length, 80 bases in length, 85 bases in length, 90 bases in length, 95 bases in length, 100 bases in length, 105 bases in length, 110 bases in length, 115 bases in length, 120 bases in length, 125 bases in length, 130 bases in length, 135 bases in length, 140 bases in length, 145 bases in length, or 150 bases in length.

In some embodiments, the fragmentation profile comprises two or more windows. The two more windows may be used to calculate a ratio, e.g., [number fragments in small window]/[number of fragments in large window]. In some embodiments, the two or more windows of a fragmentation profile are of a uniform base length size. In some embodiments, the two or more windows of a fragmentation profile comprises a first window and a second window having a different base length size. In some embodiments, the two or more windows of a fragmentation profile have a degree of overlap in a base length size.

In some embodiments, the fragments used to construct a fragmentation profile comprise fragments from a whole genome sequencing analysis. In some embodiments, the fragments used to construct a fragmentation profile comprise fragments from one or more specified locations, such as one or more chromosomes or one or more target loci or regions.

### V. Machine Learning Technique

In certain aspects, the methods described herein comprise use of a machine learning technique. In some embodiments, the machine learning technique comprises a model configured to identify, such as discover, a multimodal epigenetic signature. In some embodiments, the machine learning technique comprises a model configured to assess for the presence of a multimodal epigenetic signature.

In some embodiments, provided is a method of generating an epigenetic signature from a sample obtained from an individual, the method comprising: receiving sequencing data obtained from a non-disruptive methylation sequencing technique performed on the sample obtained from the individual; extracting features from the sequencing data, wherein the features include information from two or more of the following profiles: a methylation profile comprising information derived from one or more methylation sites; a nucleosome dynamics profile comprising information derived from any one or more of: (a) nucleosome positional information; (b) nucleosome occupancy; or (c) nucleosome fuzziness; or a fragmentation profile comprising information derived from read distributions in one or more base length windows; inputting the extracted features into a machine learning model; analyzing the features using the machine learning model to generate the epigenetic signature based on a plurality of the features; and outputting the generated epigenetic signature.

In some embodiments, provided is a method of identifying a disease epigenetic signature indicative of an individual having a disease, the method comprising: receiving sequencing data from a plurality of individuals having the disease and a plurality of individual not having the disease, wherein the sequencing data is obtained from a non-disruptive methylation sequencing technique performed on samples obtained from the individuals; extracting features from the sequencing data, wherein the features include information from two or more of the following profiles: a methylation profile comprising information derived from one or more methylation sites; a nucleosome dynamics profile comprising information derived from any one or more of: (a) nucleosome positional information; (b) nucleosome occupancy; or (c) nucleosome fuzziness; or a fragmentation profile comprising information derived from read distributions in one or more base length windows; inputting the extracted features into a machine learning model, wherein the extracted features from each of the plurality of individuals are embedded with an associated classification of the individual having the disease or not having the disease; training the machine learning model using the extracted features to identify the disease epigenetic signature; and outputting the disease epigenetic signature.

Machine learning models are known in the art, including those discussed in other sections. In some embodiments, the machine learning technique comprises a support vector machine model. In some embodiments, the machine learning technique comprises a random forest machine model. In some embodiments, the machine learning technique comprises a logistic regression machine model. In some embodiments, the input for the machine learning technique is a methylation profile, such as based on one or more qualitative and/or quantitative measures associated with one or more methylation sites. In some embodiments, the input for the machine learning technique is a nucleosome dynamics profile, such as based on a locus-specific nucleosome score based on any one or more of: (a) nucleosome positional information; (b) nucleosome occupancy; or (c) nucleosome fuzziness. In some embodiments, the input for the machine learning technique is a fragmentation profile, such as based on a fraction of nucleic acid fragments in one or more nucleic acid base length windows. In some embodiments, the input is two or more of a methylation profile, a nucleosome dynamics profile, or fragmentation profile.

In some embodiments, the methods provided herein comprises training a machine learning model. In some embodiments, the machine learning technique comprises a trained model. In some embodiments, the machine learning model is trained by inputting information obtained from any of a methylation profile, a nucleosome dynamics profile, and a fragmentation profile, wherein the information is associated with a sample having a known biological state, such as associated with a disease state or a non-disease state. In some embodiments, the machine learning model is trained using single modality data, namely, each of a methylation profile, a nucleosome dynamics profile, and a fragmentation profile. In some embodiments, the machine learning model is trained using multimodal data, namely, any combination of two or more of a methylation profile, a nucleosome dynamics profile, and a fragmentation profile. In some embodiments, training using multimodal data is according to a concatenation-based strategy, which combines multiple types of features from each sample into a single dataset for model training.

In some embodiments, the machine learning training comprises use of data from a population of individuals, such as a population of individual having a disease, e.g., a cancer, or a population of individual not having the disease, such as healthy individuals. In some embodiments, the population is 2 or more individuals, including any of 5 or more individuals, 50 or more individuals, 100 or more individuals, 500 or more individuals, or 1,000 or more individuals. In some embodiments, the individuals have a confirmed biological state, such as diagnosis of a disease, using a technique conventional in the art. In some embodiments, the methods further comprise a cross-validation procedure, such as to validate a multimodal epigenetic signature and/or the presence thereof.

### C. Diseases, Individuals, and Samples

The disclosure provided herein is useful for multimodal epigenetic signatures pertaining to a diverse array of individuals and/or diseases and/or sample types.

In some embodiments, the individual is a human. In some embodiments, the human is a male. In some embodiments, the human is a female. In some embodiments, the individual is suspected of having a disease. In some embodiments, the individual is not suspected of having a disease. In some embodiments, the individual is a healthy individual, such as an individual not having the disease.

In some embodiments, the disease is a cancer. In some embodiments, the cancer is selected from the group consisting of a prostate cancer, lung cancer, bronchial cancer, colon cancer, rectal cancer, colorectal cancer, urinary bladder cancer, melanoma, kidney cancer, renal pelvis cancer, non-Hodgkin lymphoma, oral cavity cancer, pharynx cancer, leukemia, liver cancer, intrahepatic bile duct cancer, breast cancer, uterine corpus cancer, thyroid cancer, pancreatic cancer, esophageal cancer, ovarian cancer, brain cancer, and cancer of the nervous system.

In some embodiments, the cancer comprises a primary tumor. In some embodiments, the cancer comprises one or more metastatic tumors. For example, in some embodiments, the individual has, or is suspected of having, a colon cancer with one or more metastasizes to any location such as the liver, lungs, bones, or brain.

In some embodiments, the disease is a benign inflammatory disease. In some embodiments, the disease is diverticulitis. In some embodiments, the disease is ulcerative colitis. In some embodiments, the disease is Crohn's disease. In some embodiments, the disease is infectious colitis. In some embodiments, the disease is non-infectious colitis.

In some embodiments, the sample is a cell-free DNA (cfDNA) sample. In some embodiments, the sample is a blood sample or a derivative thereof, such as plasma. In some embodiments, the sample is a blood sample, such as a whole blood sample. In some embodiments, the sample is a plasma sample. In some embodiments, the sample is a serum sample. In some embodiments, the sample is a tissue sample. In some embodiments, the sample comprises a nucleic acid originating from a tissue in an individual, such as from a diseased tissue. In some embodiments, the method further comprises obtaining a sample, such as a cfDNA sample, such as via a blood draw. In some embodiments, the method further comprises processing the sample, such as to obtain the cfDNA sample. In some embodiments, processing of the sample comprises one or more steps for separating blood components from the cfDNA.

In some embodiments, the sample is obtained from a liquid biopsy sample. In some embodiments, the liquid sample comprises blood and other liquid samples of biological origin (including, but not limited to, peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood. In some embodiments, the biological fluid is blood, a blood derivative or a blood fraction, e.g., serum or plasma. In a specific embodiment, a sample comprises a blood sample. In another embodiment, a serum sample is used. In another embodiment, a sample comprises urine. In some embodiments, the liquid sample also encompasses a sample that has been manipulated in any way after their procurement, such as by centrifugation, filtration, precipitation, dialysis, chromatography, treatment with reagents, washed, or enriched for certain cell populations.

### D. Exemplary Methods

In some aspects, provided herein is a method of determining an epigenetic signature from a sample obtained from an individual, the method comprising analyzing data obtained from a non-disruptive methylation sequencing technique performed on the sample obtained from the individual to determine the epigenetic signature, wherein the epigenetic signature comprises features obtained from two or more of the following profiles: a methylation profile comprising information derived from one or more methylation sites; a nucleosome dynamics profile comprising information derived from any one or more of: (a) nucleosome positional information; (b) nucleosome occupancy; or (c) nucleosome fuzziness; or a fragmentation profile comprising information derived from read distributions in one or more base length windows. In some embodiments, the epigenetic signature comprises features from a methylation profile and a nucleosome dynamics profile, e.g., nucleosome occupancy information.

In other aspects, provided herein is a method of generating an epigenetic signature from a sample obtained from an individual, the method comprising: receiving sequencing data obtained from a non-disruptive methylation sequencing technique performed on the sample obtained from the individual; extracting features from the sequencing data, wherein the features include information from two or more of the following profiles: a methylation profile comprising information derived from one or more methylation sites; a nucleosome dynamics profile comprising information derived from any one or more of: (a) nucleosome positional information; (b) nucleosome occupancy; or (c) nucleosome fuzziness; or a fragmentation profile comprising information derived from read distributions in one or more base length windows; inputting the extracted features into a machine learning model; analyzing the features using the machine learning model to generate the epigenetic signature based on a plurality of the features; and outputting the generated epigenetic signature. In some embodiments, the epigenetic signature comprises features from a methylation profile and a nucleosome dynamics profile, e.g., nucleosome occupancy information.

In other aspects, provided herein is a method of diagnosing a disease in an individual, the method comprising: determining an epigenetic signature from data obtained from a non-disruptive methylation sequencing technique performed on a sample obtained from the individual, wherein the epigenetic signature comprises features obtained from two or more of the following profiles: a methylation profile comprising information derived from one or more methylation sites; a nucleosome dynamics profile comprising information derived from any one or more of: (a) nucleosome positional information; (b) nucleosome occupancy; or (c) nucleosome fuzziness; or a fragmentation profile comprising information derived from read distributions in one or more base length windows; and diagnosing the disease in the individual based on the epigenetic signature as compared to a disease epigenetic signature. In some embodiments, the method further comprises diagnosing a disease in the individual based on the epigenetic signature as compared to a disease epigenetic signature. In some embodiments, the epigenetic signature comprises features from a methylation profile and a nucleosome dynamics profile, e.g., nucleosome occupancy information.

In other aspects, provided herein is a method of treating a disease in an individual, the method comprising: diagnosing the individual as having the disease according to any method provided herein; and administering an agent to treat the disease in the individual. In some embodiments, the epigenetic signature comprises features from a methylation profile and a nucleosome dynamics profile, e.g., nucleosome occupancy information.

In other aspects, provided herein is a method of identifying a disease epigenetic signature indicative of an individual having a disease, the method comprising receiving sequencing data from a plurality of individuals having the disease and a plurality of individual not having the disease, wherein the sequencing data is obtained from a non-disruptive methylation sequencing technique performed on samples obtained from the individuals; extracting features from the sequencing data, wherein the features include information from two or more of the following profiles: a methylation profile comprising information derived from one or more methylation sites; a nucleosome dynamics profile comprising information derived from any one or more of: (a) nucleosome positional information; (b) nucleosome occupancy; or (c) nucleosome fuzziness; or a fragmentation profile comprising information derived from read distributions in one or more base length windows; inputting the extracted features into a machine learning model, wherein the extracted features from each of the plurality of individuals are embedded with an associated classification of the individual having the disease or not having the disease; training the machine learning model using the extracted features to identify the disease epigenetic signature; and outputting the disease epigenetic signature. In some embodiments, the epigenetic signature comprises features from a methylation profile and a nucleosome dynamics profile, e.g., nucleosome occupancy information.

### E. Systems, Kits, and Components

In certain aspects, contemplated herein are systems, kits, and components useful for performing the methods described herein.

In some embodiments, provided herein is a system, such as a computer system, for analyzing data according to the description provided herein. In some embodiments, the system is configured to receive sequencing data. In some embodiments, the system is configured to extract features from the sequencing data, such as corresponding to any one, or combination of, a methylation profile, a nucleosome profile, or a fragmentation profile. In some embodiments, the system is configured to input the extracted features from the sequencing data into a machine learning model. In some embodiments, the system is configured to train a machine learning model. In some embodiments, the system is configured to output an epigenetic signature. In some embodiments, provided herein is a system configured to perform a computer-implemented method described herein. In some embodiments, the system comprises one or more processors, and memory storing one or more programs, the one or more programs configured to be executed by the one or more processors, and the one or more programs including instructions for performing the methods described herein. In some embodiments, the system comprises a machine learning model.

In some embodiments, provided herein is a kit, and/or components thereof, for performing aspects of the methods described herein. For example, in some embodiments, provided herein is a kit, and/or component thereof, for obtaining and/or processing a sample from an individual, such as to obtain a cfDNA sample. In some embodiments, provided herein is a kit, and/or component thereof, for performing a non-disruptive methylation sequencing technique described herein.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

### EXEMPLARY EMBODIMENTS

The following exemplary embodiments are provided herein:

Embodiment 1. A method of determining an epigenetic signature from a sample obtained from an individual, the method comprising analyzing data obtained from a non-disruptive methylation sequencing technique performed on the sample obtained from the individual to determine the epigenetic signature,
wherein the epigenetic signature comprises features obtained from two or more of the following profiles:
a methylation profile comprising information derived from one or more methylation sites;
a nucleosome dynamics profile comprising information derived from any one or more of: (a) nucleosome positional information; (b) nucleosome occupancy; or (c) nucleosome fuzziness; or
a fragmentation profile comprising information derived from read distributions in one or more base length windows.

Embodiment 2. A method of generating an epigenetic signature from a sample obtained from an individual, the method comprising:
receiving sequencing data obtained from a non-disruptive methylation sequencing technique performed on the sample obtained from the individual;
extracting features from the sequencing data,
wherein the features include information from two or more of the following profiles:
a methylation profile comprising information derived from one or more methylation sites;
a nucleosome dynamics profile comprising information derived from any one or more of: (a) nucleosome positional information; (b) nucleosome occupancy; or (c) nucleosome fuzziness; or
a fragmentation profile comprising information derived from read distributions in one or more base length windows;
inputting the extracted features into a machine learning model;
analyzing the features using the machine learning model to generate the epigenetic signature based on a plurality of the features; and
outputting the generated epigenetic signature.

Embodiment 3. A method of diagnosing a disease in an individual, the method comprising:
determining an epigenetic signature from data obtained from a non-disruptive methylation sequencing technique performed on a sample obtained from the individual,
wherein the epigenetic signature comprises features obtained from two or more of the following profiles:
a methylation profile comprising information derived from one or more methylation sites;
a nucleosome dynamics profile comprising information derived from any one or more of: (a) nucleosome positional information; (b) nucleosome occupancy; or (c) nucleosome fuzziness; or
a fragmentation profile comprising information derived from read distributions in one or more base length windows; and
diagnosing the disease in the individual based on the epigenetic signature as compared to a disease epigenetic signature.

Embodiment 4. The method of embodiment 1 or 2, further comprising diagnosing a disease in the individual based on the epigenetic signature as compared to a disease epigenetic signature.

Embodiment 5. A method of treating a disease in an individual, the method comprising:
diagnosing the individual as having the disease according to embodiment 3 or 4; and
administering an agent to treat the disease in the individual.

Embodiment 6. A method of identifying a disease epigenetic signature indicative of an individual having a disease, the method comprising:
receiving sequencing data from a plurality of individuals having the disease and a plurality of individual not having the disease,
wherein the sequencing data is obtained from a non-disruptive methylation sequencing technique performed on samples obtained from the individuals;
extracting features from the sequencing data,
wherein the features include information from two or more of the following profiles:
a methylation profile comprising information derived from one or more methylation sites;
a nucleosome dynamics profile comprising information derived from any one or more of: (a) nucleosome positional information; (b) nucleosome occupancy; or (c) nucleosome fuzziness; or
a fragmentation profile comprising information derived from read distributions in one or more base length windows;
inputting the extracted features into a machine learning model,
wherein the extracted features from each of the plurality of individuals are embedded with an associated classification of the individual having the disease or not having the disease;
training the machine learning model using the extracted features to identify the disease epigenetic signature; and
outputting the disease epigenetic signature.

Embodiment 7. The method of any one of embodiments 1-6, wherein each of the one or more methylation sites of the methylation profile are selected from the group consisting of cg18081940, cg23089825, cg16395183, cg19811148, cg07790615, cg20996351, cg04977528, cg24465685, cg20428713, cg13678973, cg25339566, cg16596317, cg23786625, cg11328303, cg19578660, cg02272851, cg10298052, cg13585930, cg23575688, cg12394201, cg08149193, cg18854419, cg07603330, cg10658542, cg13099890, cg22302985, cg13596497, cg14507533, cg25366582, cg22396555, cg10566012, cg05168229, cg10795666, cg25078444, cg16038120, cg23883632, cg18380808, cg13615592, cg00250422, cg19691260, cg16558770, cg15681853, cg03397724, cg10514097, cg06674117, cg16047279, cg12127472, cg08843809, cg08697732, cg06384763, cg04203646, cg17112426, cg08278741, cg14587524, cg26087117, cg18320766, cg08063125, cg10004780, cg18921980, cg02514318, cg20002504, cg18897632, cg15313459, cg19370054, cg16564824, cg02631468, cg01471196, cg23770904, cg18412834, cg24080247, cg11549874, cg13155421, cg19442495, cg22536150, cg05413061, cg23346462, cg09477895, cg13605674, cg13314965, cg09417547, cg00181669, cg23967169, cg10237419, cg21077559, cg27600205, cg19755714, cg18797590, cg00699993, cg06485940, cg27661394, cg00939495, cg11036833, cg23915769, cg07224726, cg02022733, cg03640756, cg15361590, cg04598517, cg06782035, cg13954457, cg25482900, cg20952257, cg14062050, cg01881524, cg11538641, cg11387340, cg05389236, cg19419054, cg10575547, cg17240815, cg24772267, cg00920327, cg00772257, cg26253500, cg23244488, cg22778435, cg26065247, cg02088996, cg19868631, cg22280038, cg07803375, cg20230721, cg03333330, cg21517947, cg10406295, cg05166490, cg07739205, cg20980783, cg06617456, cg01568998, cg13407456, cg23758305, cg20675505, cg07585876, cg03734437, and cg13410764.

Embodiment 8. The method of any one of embodiments 1-7, wherein the one or more methylation sites of the methylation profile comprise one or more gene promoter region methylation sites.

Embodiment 9. The method of any one of embodiments 1-8, wherein the methylation profile comprises quantitative information from at least one of the one or more methylation sites.

Embodiment 10. The method of embodiment 9, wherein the quantitative information is based on a (3-value from the at least one methylation sites.

Embodiment 11. The method of embodiment 9, wherein the quantitative information is based on a CHALM ratio from the at least one methylation sites.

Embodiment 12. The method of any one of embodiments 1-11, wherein the nucleosome dynamics information is based on a nucleosome at a genomic locus.

Embodiment 13. The method of any one of embodiments 1-12, wherein the nucleosome positional information is based on a window protection score (WPS).

Embodiment 14. The method of embodiment 13, wherein the WPS is an average WPS.

Embodiment 15. The method of any one of embodiments 1-14, wherein the nucleosome occupancy is based on the frequency a nucleosome occupies a genomic region.

Embodiment 16. The method of embodiment 15, wherein the nucleosome occupancy is obtained via normalized read coverage measured by counts per million.

Embodiment 17. The method of any one of embodiments 1-16, wherein the nucleosome fuzziness is based on the deviation of a nucleosome position from a prefer nucleosome position.

Embodiment 18. The method of any one of embodiments 1-18, wherein the fragmentation profile is based on one or more base length windows occupying the range of 30 to 250 bases in length.

Embodiment 19. The method of embodiment 19, wherein the base length window is at least 10 bases in length.

Embodiment 20. The method of any one of embodiments 12-17, wherein the nucleosome dynamic information is obtained via DANPOS.

Embodiment 21. The method of any one of embodiments 1-20, wherein the epigenetic signature is indicative of whether the individual has a disease.

Embodiment 22. The method of any one of embodiments 1-21, wherein the epigenetic signature comprises features from the methylation profile and the nucleosome dynamics profile.

Embodiment 23. The method of any one of embodiments 1-21, wherein the epigenetic signature comprises features from the methylation profile and the fragmentation profile.

Embodiment 24. The method of any one of embodiments 1-21, wherein the epigenetic signature comprises features from the nucleosome dynamics profile and the fragmentation profile.

Embodiment 25. The method of any one of embodiments 1-24, wherein the epigenetic signature comprises features from the methylation profile, the nucleosome dynamics profile, and the fragmentation profile.

Embodiment 26. The method of any one of embodiments 22, 24, or 25, wherein the nucleosome dynamics profile comprises information derived from nucleosome positional information.

Embodiment 27. The method of any one of embodiments 22 or 24-26, wherein the nucleosome dynamics profile comprises information derived from nucleosome occupancy.

Embodiment 28. The method of any one of embodiments 22 or 24-27, wherein the nucleosome dynamics profile comprises information derived from nucleosome fuzziness.

Embodiment 29. The method of any one of embodiments 1-28, wherein the non-disruptive methylation sequencing technique is an EM-seq technique.

Embodiment 30. The method of any one of embodiments 1-29, wherein the non-disruptive methylation sequencing technique is performed based on targeted genetic locations.

Embodiment 31. The method of any one of embodiments 1-30, further comprising performing the non-disruptive methylation sequencing technique.

Embodiment 32. The method of any one of embodiments 1-31, wherein the data obtained from the non-disruptive methylation sequencing technique comprises a plurality of sequence reads.

Embodiment 33. The method of embodiment 32, further comprising processing the plurality of sequence reads to remove low-quality reads and/or remove adaptor contamination and/or filter based on sequence read size.

Embodiment 34. The method of embodiment 32 or 33, further comprising aligning the plurality of sequence reads with a reference genome.

Embodiment 35. The method of any one of embodiments 2 or 6-34, wherein the machine learning model comprises a support vector machine model, a random forest machine model, or a logistic regression machine model.

Embodiment 36. The method of embodiment 35, further comprising a cross-validation procedure.

Embodiment 37. The method of any one of embodiments 1-22, wherein the sample is a cell-free DNA sample.

Embodiment 38. The method of any one of embodiments 1-23, further comprising obtaining the sample.

Embodiment 39. The method of any one of embodiments 1-24, wherein the disease is a cancer.

Embodiment 40. The method of embodiment 39, wherein the cancer is a colorectal cancer.

Embodiment 41. The method of any of embodiments 1-40, wherein the individual is a human.

Embodiment 42. The method of any one of embodiments 1-41, wherein the individual is suspected of having a disease.

### EXAMPLES

### Example 1

### A method for a Multimodal Epigenetic Sequencing Assay (MESA) for Accurate Detection of Human Cancer

This example describes a method for a multimodal epigenetic sequencing assay (MESA) for accurate detection of human cancer. The method demonstrated herein is a flexible and sensitive method capable of combining at least two profiles (such as selected from a methylation profile, a nucleosome dynamics profile, and a fragmentation profile) in a single assay using non-disruptive enzymatic methylation sequencing and innovative bioinformatics algorithms.

Plasma cell-free DNA (cfDNA) are degraded DNA fragments released to the blood stream. In healthy individuals, plasma cfDNA is mainly derived from the apoptosis of normal hematopoietic cells, with minimal contributions from other tissues. In individuals with specific physiological or disease conditions, a fraction of cfDNA may have different origins, such as diseased tissue, when compared to the healthy state.

A frequently reported epigenetic change for cancer cells is DNA methylation, which can occur early in tumorigenesis. Bisulfite genomic sequencing is regarded as the gold standard technology for DNA methylation detection. However, bisulfite treatment is harshly damaging to DNA, thus imperfectly capturing the cfDNA methylome and biasing the downstream study of potential biomarkers.

In the present study, we utilized a recently developed bisulfite-free DNA methylation sequencing method that utilizes non-destructive enzymes. As demonstrated below, we found that the non-destructive nature of enzymatic methylation sequencing also enables additional epigenetic analysis (e.g., fragmentation profile or nucleosome dynamics profile comprising nucleosome position, nucleosome occupancy, and nucleosome fuzziness) simultaneously in cfDNA methylation sequencing analysis. Although the nucleosome organization is weakly related to the fragmentation profile, nucleosome information can provide information other than fragmentation. Nucleosome organization focus on the position-specific cfDNA fragment, while fragmentation profile only focuses on the size of the cfDNA fragments globally. Even if two samples have the same fragment size distribution, they can still have very different nucleosome organization in most regions. Furthermore, fragmentation profile normally requires whole genome sequencing, whereas nucleosome organization is suitable for targeted sequencing with small regions (e.g., 2 kb).

Here, we demonstrate a three-in-one method of measuring cfDNA to obtain a methylation profile, nucleosome dynamics profile, and fragmentation profile in a single assay using non-disruptive enzymatic methylation sequencing and highly innovative bioinformatics algorithms. Integrated analysis of these multimodal features significantly improved the accurate detection of colon cancer. We designed an enzymatic-based target cfDNA methylation sequencing panel for 83 colon cancer patients and 83 healthy individuals using an EM-seq technique. The target regions included both a commercially available Twist Methylome panel and a custom nucleosome organization panel including open chromatin ATAC peaks, CpG islands, enhancers, transcription start sites (TSS), RNA splicing sites, and polyadenylation sites (PAS) of cancer genes.

Raw sequencing data were first trimmed by TrimGalore to remove low-quality reads and potential adaptor contamination. Then, the remaining sequencing reads were aligned to the hg19 human genome reference using BSMAP. The aligned reads were further processed by Samtools and Bedtools to only keep primarily mapped reads with fragment sizes between 80 by and 200 bp. This final bam file served as the input file for all the following processes.

Using this cfDNA data, we extracted three types of features from a single assay: a methylation profile, a nucleosome dynamics profile, and a fragmentation profile. Specifically, for the methylation profile, conventional mean methylation (beta values) of the target methylations sites was performed. Using Methratio.py (BSMAP), we extracted the methylation ratio from aligned barn files for the target methylation sites. Additionally, CHALM methylation analysis was performed according to Xu et al. (Nature Communication, 2021). For the nucleosome dynamics profile, three features were assessed—nucleosome positional information (via a windows protection score; WPS), nucleosome occupancy, and nucleosome fuzziness. Window protection score (WPS) is used to assess position via the concept that cfDNA fragment endpoints should cluster around nucleosome boundaries and be depleted on the nucleosome itself. WPS was calculated as the number of complete fragments minus the number of fragment endpoints within a given window size. The average WPS for each sliding window described herein was calculated. Nucleosome occupancy reflects the frequency with which nucleosomes occupy a given DNA region in a cell population. We split each 2 kb target region into 500 or 1000 bp sliding windows with 10 bp steps. Then, for each sliding window, we calculated nucleosome occupancy features in two ways: (1) Normalized read coverage measured by counts per million (CPM) using bamCoverage tool from deepTools; and (2) Occupancy values reported by DANPOS2. In a cell population, the exact positions of the nucleosome in each DNA region may deviate from a most preferred position. Fuzziness score is defined as the deviation of nucleosome positions within the region in a cell population. For each sliding window described above for nucleosome occupancy, we calculated the average fuzziness score (reported by DANPOS2) of all the nucleosomes whose center is located within the window. The fragmentation profile was defined as the fraction of cfDNA fragments in a specific size range. The features included P(80-150), P(80-155), P(80-160), P(80-165), P(125-155), P(170-175), P(170-200), P(175-200), P(80-150)/P(151-200), P(80-155)/P(156-200), P(80-160)P(161-200) and P(80-165)/P(166-200). Here, P(x-y) is the fraction of fragments in a size range from x to y bp. We extracted two sets of these features in different scales. For the first set, we calculated fragmentation profiles by combining all the targeted regions. For the second set, we calculated fragmentation profiles for each chromosome, respectively.

We trained and tested a machine learning cancer detection model with either single modality data, i.e., each of the three types of features, or multimodal, i.e., any combination of three types of features. We integrate multimodal data with a concatenation-based strategy. This strategy combines multiple types of features from each sample into one single dataset for model training. We used three machine learning algorithms: support vector machines, random forest, and logistic regression. These algorithms were trained and evaluated using the following cross-validation procedure: In each iteration, the samples were split into 70%-30% training-testing sub-datasets. Then the area under the receiver operator characteristic curve (AUC-ROC) and sensitivity were calculated accordingly.

Here, we showed an example result from a multimodal model based on the combination of conventional mean methylation and nucleosome occupancy reported by DANPOS2. The multimodal model was trained using the random forest algorithm. The median values of 50 iterations are shown in Table 1. From the result, we first find that both DNA methylation and nucleosome occupancy alone are sensitive predictors of colon cancer in patient cfDNA. Further, nucleosome occupancy plus methylation outperforms either single model alone, showing significant improvements using the multimodal approach.

TABLE 1
Summary of model performance for detection of colon cancer.

| | | Sensitivity at 95% | Sensitivity at 90% |
|---|---|---|---|
| Feature type | AUC | specificity | specificity |
| Methylation | 0.850 | 0.500 | 0.568 |
| Nucleosome | 0.847 | 0.409 | 0.591 |
| occupancy (1 kb) | | | |
| Methylation plus | 0.879 | 0.545 | 0.682 |
| nucleosome | | | |
| occupancy (1 kb) | | | |

The combination of a multimodal approach and our innovative bioinformatics algorithms provide a significant advancement to the field of cfDNA liquid biopsy cancer detection. In addition to the demonstration herein, another advantage of the multimodal assay is its flexibility. Each of the three modalities of epigenomic information can be included or excluded from a prediction model. For example, cancer types in which nucleosome occupancy is relatively unchanged may benefit only from the integration of the remaining two modalities, methylation and fragmentation. Removal of nucleosome organization in this case could prevent confounding and unnecessary complexity. This multimodal approach allows for the development of an unbiased combinatorial prediction model. All three modalities are simultaneously captured in a single targeted sequencing assay, offering full flexibility without wasting a multiplex assay.

1. A method of determining an epigenetic signature from a sample obtained from an individual, the method comprising analyzing data obtained from a non-disruptive methylation sequencing technique performed on the sample obtained from the individual to determine the epigenetic signature,
wherein the epigenetic signature comprises features obtained from two or more of the following profiles:
a methylation profile comprising information derived from one or more methylation sites;
a nucleosome dynamics profile comprising information derived from any one or more of: (a) nucleosome positional information; (b) nucleosome occupancy; or (c) nucleosome fuzziness; or
a fragmentation profile comprising information derived from read distributions in one or more base length windows.

2. A method of generating an epigenetic signature from a sample obtained from an individual, the method comprising:
receiving sequencing data obtained from a non-disruptive methylation sequencing technique performed on the sample obtained from the individual;
extracting features from the sequencing data,
wherein the features include information from two or more of the following profiles:
a methylation profile comprising information derived from one or more methylation sites;
a nucleosome dynamics profile comprising information derived from any one or more of: (a) nucleosome positional information; (b) nucleosome occupancy; or (c) nucleosome fuzziness; or
a fragmentation profile comprising information derived from read distributions in one or more base length windows;
inputting the extracted features into a machine learning model;
analyzing the features using the machine learning model to generate the epigenetic signature based on a plurality of the features; and
outputting the generated epigenetic signature.

3-5. (canceled)

6. A method of identifying a disease epigenetic signature indicative of an individual having a disease, the method comprising:
receiving sequencing data from a plurality of individuals having the disease and a plurality of individual not having the disease,
wherein the sequencing data is obtained from a non-disruptive methylation sequencing technique performed on samples obtained from the individuals;
extracting features from the sequencing data,
wherein the features include information from two or more of the following profiles:
a methylation profile comprising information derived from one or more methylation sites;
a nucleosome dynamics profile comprising information derived from any one or more of: (a) nucleosome positional information; (b) nucleosome occupancy; or (c) nucleosome fuzziness; or
a fragmentation profile comprising information derived from read distributions in one or more base length windows;
inputting the extracted features into a machine learning model,
wherein the extracted features from each of the plurality of individuals are embedded with an associated classification of the individual having the disease or not having the disease;
training the machine learning model using the extracted features to identify the disease epigenetic signature; and
outputting the disease epigenetic signature.

7. The method of claim 1, wherein each of the one or more methylation sites of the methylation profile are selected from the group consisting of cg18081940, cg23089825, cg16395183, cg19811148, cg07790615, cg20996351, cg04977528, cg24465685, cg20428713, cg13678973, cg25339566, cg16596317, cg23786625, cg11328303, cg19578660, cg02272851, cg10298052, cg13585930, cg23575688, cg12394201, cg08149193, cg18854419, cg07603330, cg10658542, cg13099890, cg22302985, cg13596497, cg14507533, cg25366582, cg22396555, cg10566012, cg05168229, cg10795666, cg25078444, cg16038120, cg23883632, cg18380808, cg13615592, cg00250422, cg19691260, cg16558770, cg15681853, cg03397724, cg10514097, cg06674117, cg16047279, cg12127472, cg08843809, cg08697732, cg06384763, cg04203646, cg17112426, cg08278741, cg14587524, cg26087117, cg18320766, cg08063125, cg10004780, cg18921980, cg02514318, cg20002504, cg18897632, cg15313459, cg19370054, cg16564824, cg02631468, cg01471196, cg23770904, cg18412834, cg24080247, cg11549874, cg13155421, cg19442495, cg22536150, cg05413061, cg23346462, cg09477895, cg13605674, cg13314965, cg09417547, cg00181669, cg23967169, cg10237419, cg21077559, cg27600205, cg19755714, cg18797590, cg00699993, cg06485940, cg27661394, cg00939495, cg11036833, cg23915769, cg07224726, cg02022733, cg03640756, cg15361590, cg04598517, cg06782035, cg13954457, cg25482900, cg20952257, cg14062050, cg01881524, cg11538641, cg11387340, cg05389236, cg19419054, cg10575547, cg17240815, cg24772267, cg00920327, cg00772257, cg26253500, cg23244488, cg22778435, cg26065247, cg02088996, cg19868631, cg22280038, cg07803375, cg20230721, cg03333330, cg21517947, cg10406295, cg05166490, cg07739205, cg20980783, cg06617456, cg01568998, cg13407456, cg23758305, cg20675505, cg07585876, cg03734437, and cg13410764.

8. The method of claim 1, wherein the one or more methylation sites of the methylation profile comprise one or more gene promoter region methylation sites.

9. The method of claim 1, wherein the methylation profile comprises quantitative information from at least one of the one or more methylation sites.

10. The method of claim 9, wherein the quantitative information is based on a β-value from the at least one methylation sites or wherein the quantitative information is based on a CHALM ratio from the at least one methylation sites.

11. (canceled)

12. The method of claim 1, wherein the nucleosome dynamics information is based on a nucleosome at a genomic locus.

13. The method of claim 1, wherein the nucleosome positional information is based on a window protection score (WPS).

14. (canceled)

15. The method of claim 1, wherein the nucleosome occupancy is based on the frequency a nucleosome occupies a genomic region.

16. (canceled)

17. The method of claim 1, wherein the nucleosome fuzziness is based on the deviation of a nucleosome position from a prefer nucleosome position.

18. The method of claim 1, wherein the fragmentation profile is based on one or more base length windows occupying the range of 30 to 250 bases in length.

19-21. (canceled)

22. The method of claim 1, wherein the epigenetic signature comprises features from:
the methylation profile and the nucleosome dynamics profile
ii) the methylation profile and the fragmentation profile;
iii) the nucleosome dynamics profile and the fragmentation profile; or
iv) the methylation profile, the nucleosome dynamics profile, and the fragmentation profile.

23-25. (canceled)

26. The method of claim 22, wherein the nucleosome dynamics profile comprises information derived from nucleosome positional information, nucleosome occupancy, nucleosome fuzziness, or a combination thereof.

27-28. (canceled)

29. The method of claim 1, wherein the non-disruptive methylation sequencing technique is an EM-seq technique.

30. The method of claim 1, wherein the non-disruptive methylation sequencing technique is performed based on targeted genetic locations.

31. The method of claim 1, further comprising performing the non-disruptive methylation sequencing technique.

32. The method of claim 1, wherein the data obtained from the non-disruptive methylation sequencing technique comprises a plurality of sequence reads.

33-36. (canceled)

37. The method of claim 1, wherein the sample is a cell-free DNA sample.

38. (canceled)

39. The method of claim 1, wherein the disease is a cancer.

40-42. (canceled)
